(19) United States
(12) Patent Application Publication
(10) Pub. No.: US 2012/0021407 A1
(43) Pub. Date: Jan. 26, 2012

(54) Methods and Devices for Rapid Urine Concentration

(75) Inventor: Yousef Haj-Ahmad, St. Catharines (CA)

(73) Assignee: Norgen Biotek Corp., Thorold (CA)

(21) Appl. No.: 13/188,583

(22) Filed: Jul. 22, 2011

(30) Foreign Application Priority Data
Jul. 23, 2010 (CA) 2,710,904

(51) Int. Cl. G01N 33/53; C12Q 1/68; C12Q 1/70; C12M 1/34

(52) U.S. Cl. 435/5; 422/69; 435/287.2; 436/501; 435/6.12; 435/7.92

(57) ABSTRACT

The present invention provides a device for the concentration of one or more target analytes contained in a urine sample. The device comprises a tube comprising an upper portion defining an opening for receiving the urine sample and a lower tapered portion terminating in collection reservoir. The tube contains a predetermined amount of a particulate binding agent which specifically binds the one or more target analytes and of a predetermined amount of a binding buffer. The device comprises means for seating the opening of the tube. The present invention further provides methods and kits for concentrating one or more target analytes in murine sample.

### RELATED APPLICATION

This application claims priority under 35 U.S.C. §119 to Canadian patent application No. 2,710,904 filed Jul. 23, 2010, the contents of which are incorporated by reference.

### FIELD OF INVENTION

The present invention relates to methods and devices useful for the collection and concentration of urine samples.

### BACKGROUND

Recently there has been a shift towards non-invasive biological sample collection for research and diagnostics. Non-invasive specimen collection has a number of advantages including the fact that it is preferred by patients, requires less specialized personnel and equipment, and can be performed in various different settings, not just doctor's offices (Cook et al., 2005).

Urine represents an ideal non-invasive sample for both research and diagnostic applications. It has recently been well established that urine is a rich source of macromolecules including DNA, RNA and proteins, and the diagnosis of STI's based on the presence of DNA or microorganisms within the urine is well documented (Chapin, 2006). Urine DNA can be used in many different applications in diagnostics, including monitoring graft rejection (Zhang et al., 1999), transplantation monitoring (Botezatu et al, 2000), detecting nephrotoxicity (Le Lann et al., 1994), and even prenatal sex determination (Botezatu et al., 2000). Urine DNA is also being used in the detection of pathogenic bacteria (Shigemura et al., 2005), the Lyme disease-associated bacteria Borrelia burgdorferi (Exner et al., 2003), detection of HPV (Prusty et al., 2005), CMV (Paixo et al., 2005), BK virus and SV40 (Vanchiere et al., 2005). Furthermore, a variety of studies have shown that different types of cancer can be detected using urine derived-DNA, including bladder cancer (Pirskalaishvili et at, 1999; Su et al,, 2004), and prostate cancer (Henrique et al., 2004). Also, urine-derived DNA has been used in identity testing (Marques et al., 2005). Furthermore, DNA. arising from cells shed into the lumen of genitourinary tract can be used for the detection of genetic anomalies and neoplasia associated with the bladder, prostate or kidney (Cairns, 2004; Mehta et at, 2006), The use of urine as a sample for the diagnosis of non-genitourinary infections has also been successfully reported in tuberculosis (Cannas et al, 2008), leishmaniasis (Fisa et al., 2008) and malaria (Mharaturwa et al., 2006). Thus, urine can be used for point-of-care diagnosis of STI's as well as many other diseases and infections. Therefore the collection, concentration and/or preservation of urine samples for various downstream applications and tests are necessary.

Traditionally, urine samples have been collected into simple, plastic, screw-top collection containers. Shipping large volumes of urine can be problematic and costly, especially, if the urine must be shipped on ice or under cold temperature,

Another problem associated with the use of urine for diagnostic and research applications is that the analytes present within the urine are often very dilute or present in very small amounts, For example, lipoarabinomannan (LAM), a major glycolipid component of the cell wall of Mycobacterium tuberculosis, the causative agent of Tuberculosis, can be found in dilute amounts in urine. Traditional methods for the detection of LAM in urine for diagnosis of Tuberculosis require concentration and purification of the urine, which is very time-consuming (Reither et al., 2009). Thus concentration of the urine sample and. concentration of the analytes present within the urine must be performed prior to analyzing the urine sample. Common methods for the concentration of urine samples involve the use of filters or membranes, These are often molecular-weight cut-off membranes and they require the use of centrifugation in order to concentrate the sample. There would be problems with using these concentration devices in the field or in resource-limited areas to concentrate the components of the urine for analysis, such as in the case of point-of-care diagnostic tests. Furthermore, these concentration steps often take 2-4 hours to complete. During such an extended period of time microorganisms present in urine would grow thus altering the composition of urine. Therefore, the combination of these drawbacks, including problems with shipping and concentration, greatly limit the use of urine for diagnostics, particularly in resource-limited settings and for point-of-care diagnostics.

### SUMMARY OF INVENTION

The present invention provides a method that allows for the rapid concentration of urine samples that can be performed without the use of specialized equipment, such as filters or centrifuges, or the need for electricity,

In one aspect, provided is a method for concentrating one or more target analytes in a urine sample comprising the steps of:
a) providing a concentration device comprising a tube comprising an upper portion defining an opening for receiving the urine sample and a lower tapered portion terminating in a collection reservoir; said tube containing a predetermined amount of a particulate binding agent which specifically binds the one or more target analytes and of a predetermined amount of a binding buffer; and means for sealing the opening of the tube;
b) collecting a sample of urine into the concentration device;
c) mixing the urine sample, binding agent, and binding solution;
d) allowing the binding agent to settle within the container by gravity; and
e) removing the liquid portion of the urine sample from the concentration device, wherein the binding agent remaining in the concentration device is bound with the one or more target analytes,

In an embodiment, the binding agent comprises silicon carbide.

In an embodiment, the one or more analytes is DNA, RNA, microRNA, protein, bacteria, virus, yeast, exfoliated cells, polycyclic aromatic compounds and/or lipopolysaccharides.

In an embodiment, the method further comprises the step of:

f) adding a urine preservation solution to the binding agent bound with the one or more target analytes.

In an embodiment, the preservation solution is Norgen Urine Preservative™.

In an embodiment, the method further comprises the step of adding a lysis solution to the binding agent bound with the one or more target analytes.

The present invention further provides devices that allow for the rapid concentration of urine samples and which do not require the use of electricity.

In another aspect, provided is a device for the concentration of one or more target analytes contained in a urine sample, said device comprising:
a tube comprising an upper portion defining an opening for receiving the urine sample and a lower tapered portion terminating in a collection reservoir; said tube containing a predetermined amount of a particulate binding agent which specifically binds the one or more target analytes and of a predetermined amount of a binding buffer; and
means for sealing the opening of the tube.

In an embodiment, the collection reservoir is substantially parallelepipedal in cross-section,

In an embodiment, the collection reservoir comprises a tapered lower portion.

In an embodiment, the collection reservoir has a volume sufficient to contain the pre-determined amount of the particulate binding agent.

In an embodiment, the binding agent comprises silicon carbide.

In an embodiment, the means for sealing the opening of the tube is a screw on cap.

In another aspect, provided is a device for the concentration of one or more target analytes contained in a urine sample, said device comprising:
a first tube and a second tube,
said first tube comprising an upper portion defining an opening for receiving the urine sample and a lower portion for releasably attaching an upper portion of the second tube; wherein when the first tube and the second tube are attached to one another, the interior of said first tube and the interior of said second tube are in fluid communication;
said second tube comprising lower tapered portion terminating in a collection reservoir; said second tube containing a predetermined amount of a particulate binding agent which specifically binds the one or more target analytes and of a predetermined amount of a binding butler; and
means for sealing the opening of the first tube.

In an embodiment, the binding agent comprises silicon carbide.

In an embodiment, the device further comprises means for sealing the opening of the second tube.

In an embodiment, the means for sealing the opening of the second tube is a screw on cap.

In an embodiment, the means for sealing the opening of the first tube is a screw on cap.

In an embodiment, the lower portion of the first tube and the upper portion of the second tube are provided with matching threads, whereby the lower portion of the first tube and the upper portion of the second tube can be screwed together.

In an embodiment, the collection reservoir is substantially parallelepipedal in cross-section.

In an embodiment, the collection reservoir comprises a tapered lower portion.

In an embodiment, the collection reservoir has a volume sufficient to contain the pre-determined amount of the particulate binding agent.

In a further aspect, provided is a kit for the collection and concentration of one or more target analytes from a urine sample; said kit comprising:
the concentration device according to the present invention; and
instructions for use of said concentration device.

In an embodiment, the kit further comprises a pre-aliquoted sealed ampule containing a preservation solution.

In an embodiment, the preservation solution is Norgen Urine Preservative™.

In an embodiment, the pre-aliquoted sealed ampule is a dropper, a syringe, a micro-doser, a pipette, a vial, or a pouch.

In a further embodiment, the kit further comprises a lysis solution.

### DESCRIPTION OF THE DRAWINGS

Preferred embodiments of the invention will now be described, by way of example, with reference to the accompanying drawings, in which:

FIG. 1 is perspective view of a first embodiment of an urine concentration device without an outer housing;

FIG. 2 is a front view of the urine concentration device of FIG. 1;

FIG. 3A is a side view of the urine concentration device of FIG. 1;

FIG. 3B is a cross-sectional side view of the urine concentration device of FIG. 1;

FIG. 4A is an exploded side view of the urine concentration device of FIG. 1 with an outer housing;

FIG. 4B is an exploded cross-sectional side view taken along tine A-A of the urine concentration device of FIG. 4A;

FIG. 5A is a side view of the urine concentration device of FIG. 1 with an outer housing;

FIG. 5B is a cross-sectional side view taken along line A-A of the urine concentration device of FIG. 5A;

FIG. 6A is an exploded side view of a second embodiment of an urine concentration device;

FIG. 613 is an exploded cross-sectional side view taken along tine A-A of the urine concentration device of FIG. 6A;

FIG. 7A is a side view of the second embodiment of an urine concentration device;

FIG. 7B is an exploded cross-sectional side view taken along line A-A of the urine concentration device of FIG. 7A;

FIG. 8 is a bar graph showing the amount of protein isolated from concentrated urine samples (E1 and E2);

FIG. 9 is a gel image showing the DNA isolated from concentrated urine samples (Lane 2) and the decanted urine supernatant (Lane 1);

FIG. 10 is a bar graph showing that increasing ethanol concentrations allow silicon carbide to bind and concentrate increasing amounts of LAM from urine;

FIG. 11 is a bar graph showing that increasing guanidine hydrochloride concentrations allow silicon carbide to bind and concentrate increasing amounts of LAM from urine; and

FIG. 12 is a bar graph showing that increasing sodium citrate concentrations allow silicon carbide to bind and concentrate increasing amounts of LAM from urine.

Similar references are used in different figures to denote similar components.

### DESCRIPTION

Conventional methods for urine collection do not allow for the rapid concentration of urine samples without the requirement for special equipment (i.e. filters and centrifuges) or electricity, and as such, are not suitable for use in resource-limited settings or for point-of-care diagnostics.

In contrast, the concentration devices and methods disclosed herein rapidly and simply concentrate macromolecules, micro-organisms and cells found in urine without the necessity for additional specialized equipment or electricity and are well suited for use in resource-limited areas. The disclosed methods and devices can be used for both research and diagnostic applications, and offers considerable advantages over any other current urine collection and concentration methods and systems. The disclosed methods and devices are useful for reducing the shipping volume for urine samples as the urine samples can be concentrated as high as 50-fold to 100-fold. Furthermore, with the use of a chemical preservative, the concentrated urine analytes can be shipped at room temperature. This makes shipping urine samples much easier as refrigeration is not required and the sample volumes shipped are very small.

In a first aspect, provided is a method for concentrating one or more target analytes in a urine sample comprising the steps of: a) collecting a sample of urine into a concentration device comprising at least a container and a particulate binding agent; b) contacting the urine sample with the particulate binding agent in the presence of a binding buffer, wherein said binding agent specifically binds the one or more target analytes; c) mixing the urine sample, binding agent, and binding buffer; d) allowing the binding agent to settle within the container by gravity; and e) removing the liquid portion of the urine sample from the container, wherein the binding agent remaining in the container is bound with the one or more target analytes.

The choice of binding agent and binding buffer will depend on the desired urine analyte to be concentrated. The one or more target analytes may include macromolecules (including but not limited to DNA, RNA, microRNA, protein, polycyclic aromatic compounds, lipopolysaccharides), microorganisms (including but not limited to bacteria, fungi, yeast), viruses and cells (including but not limited to exfoliated cells). Particulate binding agents capable of binding macromolecules, microorganisms and/or cells are well known in the art and those which can be precipitated or settled by gravity are suitable for the methods disclosed herein, In one embodiment, the binding agent may be a resin wherein the surface of the resin is modified by methods known in the art in order to specifically bind the target analytes in the urine sample.

In a preferred embodiment, the binding agent comprises silicon carbide (SiC). Silicon carbide is available in a variety of grit sizes or grades, and each grade has a different average particle size. In a preferred embodiment, the binding agent will comprise silicon carbide having a grit size between 1000-2500 (diameter ca. 1-5 um), preferably a grit size between 2000-2500 and even more preferably, a grit size of 2000.

In embodiments employing silicon carbide, the binding buffers may comprise about 0.1 to 1 mM phosphoric acid, preferably about 0.5 mM phosphoric acid, and more preferably about 0.833 MM phosphoric acid.

The amount of binding agent and binding buffer used will depend on the volume of the urine sample and the predicted amount of the target analyte contained in the urine sample and can be determined by the person skilled in the art using conventional methods. In embodiments employing silicon carbide, typically, for a urine sample of about 20-30 ml, the amount of silicon carbide will be about 0.3-0.5 g and the amount of binding buffer will be about 0.5-1 ml.

The urine sample, binding agent, and binding buffer can be mixed by sealing a container containing the urine sample, binding agent, and binding buffer and shaking the container. After mixing, the binding agent settles rapidly by gravity to the bottom of the container. In embodiments where the binding agent comprises silicon carbide, the settling time is typically about 10 minutes. It will be appreciated however, that the binding agent may take more or less time to settle depending on the amount of binding agent used and the volume of sample. After the binding agent has settled, the liquid can be removed from the container by decanting. Alternatively, the liquid can be removed through the use of a pipette, syringe, dropper or any other device capable of transferring liquid. The binding agent remaining in the container is bound with the one or more target analytes.

The container containing the binding agent and target analyte(s) can be sealed for storage. it is generally recommended that urine samples be tested within two hours of collection. If testing is not performed within 2 hours, the urine samples may be placed in a refrigerator for short term preservation of the urine sample. However, during field studies and in resource-limited areas a refrigerator may not be available to allow for storage of the urine sample. Thus, chemical preservation of urine specimens may be utilized if testing or refrigeration within that two hour window is not possible. In a further embodiment, prior to storage and/or further processing of the target analytes, a urine preservation solution is added to the binding agent bound with the one or more target analytes.

A variety of urine preservatives (e.g, tartaric and boric acids) are available that allow urine to be kept at room temperature. These preservatives are designed to maintain the specimen in a state equivalent to refrigeration by inhibiting the proliferation of organisms that could result in a false positive culture or bacterial overgrowth. Generally, the length of preservation capacity ranges from 24 to 72 hours. A preservation solution can also be used to preserve the integrity of the target analytes (such as for example, DNA, RNA and proteins) at room temperature. The preservation solution may also kill concentrated microorganisms bound to the binding agent thereby resulting in the microorganism being non-infectious.

In some embodiments, it may be desirable to provide a preservation solution which also allows for preservation of the nucleic acids and proteins at room temperature for storage or shipping. While it will be appreciated that the time period for preservation may be as short as the time necessary to transfer a sample from the point of collection to the point of analysis, extended periods of preservation may be also be desirable (i.e. days, months or greater). In a preferred embodiment, the method disclosed herein employs NORGEN URINE PRESERVATIVE ™ (Norgen's Urine Preservative Single Dose Ampules, Thorold, Canada, Cat# 18124) as the preservative. The use of NORGEN URINE PRESERVATIVE™ allows for the preservation of the concentrated analytes for about 1 year at room temperature. While this preservation solution can be used to preserve samples for room temperature storage and shipping, which is generally from 15° C. to 40″C, in other embodiments the samples may be stored in cool environments, such as −20° C. or 4° C., or in warm environments including up to about 55° C.

In another embodiment, the method disclosed herein further comprises the step of adding a lysis solution to the binding agent bound with the one or more target analytes. The method can be practiced using conventional lysis solutions known in the art such as, but not limited to lysis solutions comprising lytic enzymes, detergents and/or chaotropes. The lysis solution will lyse the concentrated microorganisms and/or cells such that the nucleic acids and proteins are released. Once lysed the released nucleic acids and proteins can be isolated using methods known in the art for research or diagnostic purposes. The isolated nucleic acids can then be analyzed by any technique known in the art which can be used for analyzing nucleic acids, including but not limited to gel electrophoresis or PCR amplification techniques while the isolated proteins can be analyzed by any technique known in the art which can be used for analyzing proteins, including but not limited to immunoassays.

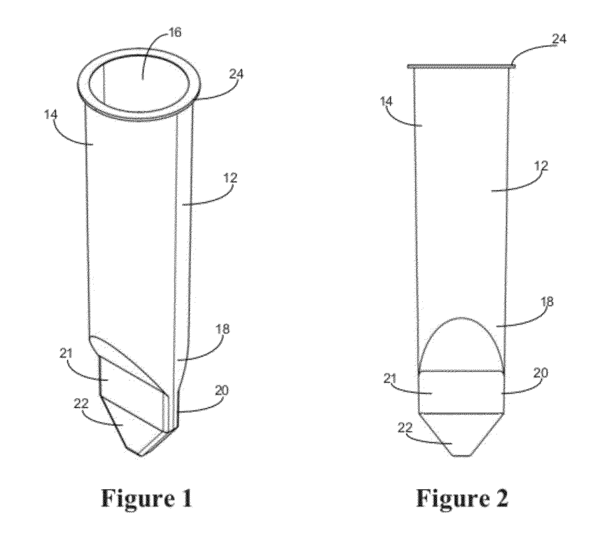

FIGS. 1, 2, 3A-B, 4A-B, and 5A-B illustrate a first embodiment of a device 10 for the concentration of one or more target analytes contained in a urine sample and which can be used to practice the method disclosed above, The device 10 comprises a tube 12 which comprises an upper portion 14 defining an opening 16 for receiving the urine sample and a lower tapered portion 18 terminating in a collection reservoir 20. Preferably, the collection reservoir 20 is substantially parallelepipedal in cross-section. The collection reservoir 20 may comprise an upper portion 21 and a tapered lower portion 22 as shown in FIG. 1.

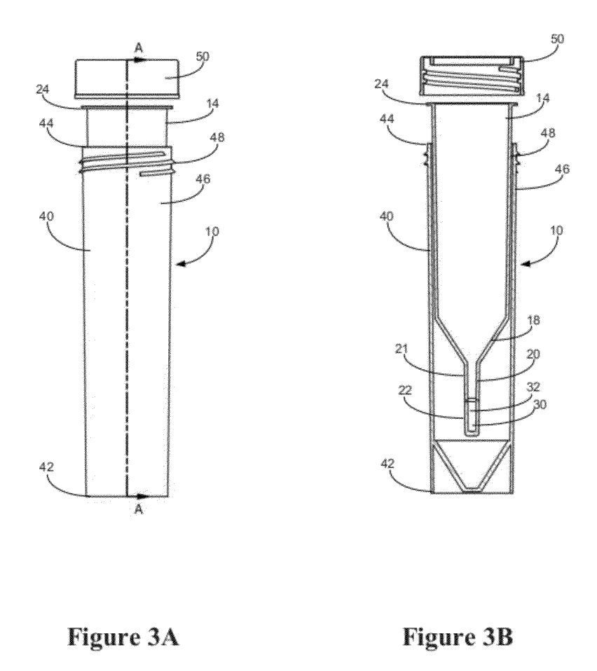

As shown in FIG. 3, the tube 12 contains a predetermined amount of a binding agent 30 which specifically binds the one or more target analytes and a predetermined amount of a binding buffer 32. In use, the collection reservoir 20 of the tube 12 aids with the decanting of resulting supernatant following concentration, without the loss of the binding agent 30. As the collection reservoir 20 is constricted in width relative to the upper portion 14 of the tube 12, the binding agent 30 is retained in the collection reservoir 20 when the tube 12 is tipped to decant the unbound liquid portion of the urine sample. Furthermore, by constricting the width of the collection reservoir 20, any liquid remaining in the collection reservoir 20 can be easily removed using a pipette or other similar device. Constricting the width of the collection reservoir 20 raises the level of a volume of fluid, relative to the level of the same volume of fluid in a container having a greater width, thereby facilitating access to the fluid.

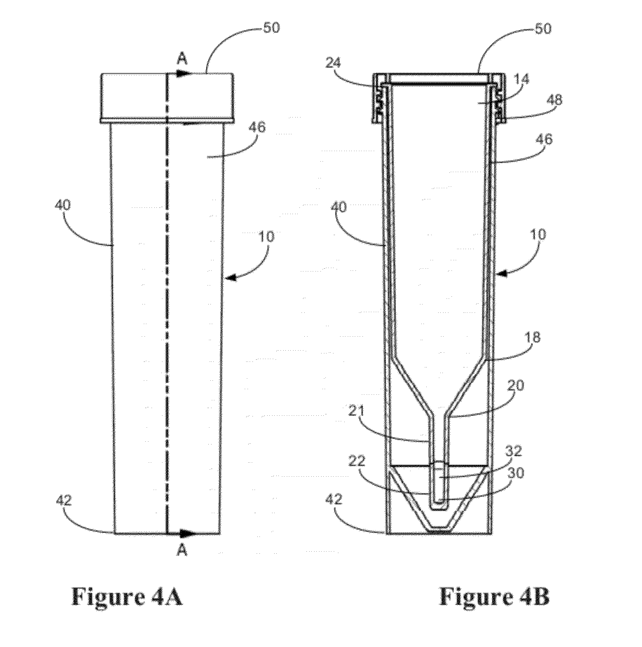

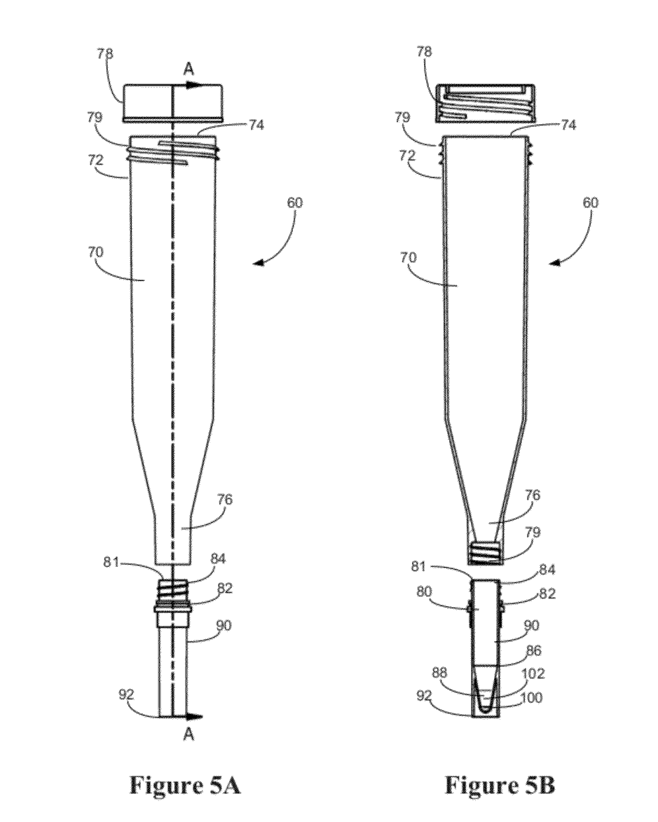

As shown in FIGS. 4A-B and 5A-B, tube 12 may be contained in an outer housing 40. The outer housing is sized and shaped to contain tube 12 with the upper portion 46 defining an opening. Preferably, the outer housing 40 has a flat bottom edge 42 which allows the device 10 to stand freely without the use of an external support. The upper portion 14 of tube 12 may be provided with a flange 24 which overlaps the outer peripheral edge 44 of the upper portion 46 of the outer housing 40 to allow the tube 12 to be supported within the outer housing 40. Device 10 further comprises means for sealing 50 the opening of the tube 12. In one embodiment, the upper portion 46 of the outer housing 40 is provided with threads 48 for receiving a screw on cap 50 for sealing the device 10.

The device 10 may be constructed using polypropylene or other suitable materials. The size of device 10 will depend on the amount of urine to be collected. In a preferred embodiment, the opening of the tube 12 can have an outer diameter of about 26.5 mm and an inner diameter of about 24.5 mm. The tapered lower portion 18 of the tube 12 can have an outer diameter of about 5.0 mm and an inner diameter of about 3.0 mm. The tube 12 can be about 110 mm in length wherein, the upper portion 14 is about 70 mm in length, the lower tapered portion 18 is about 15 mm in length, and an upper portion 21 of the collection reservoir 20 is about 12 mm in length and a lower tapered portion 22 of the collection reservoir 20 is about 14 mm in length. The volume of the tube 12 can be about 25 ml. The volume of the collection reservoir 20 can be about 1.1 ml.

As discussed above, choice of binding agent and binding buffer will depend on the desired urine analyte to be concentrated. In embodiments, wherein the analyte specific resin is silicon carbide, the binding buffer may comprise about 0.1 to 1 mM phosphoric acid, preferably about 0.5 mM phosphoric acid, and more preferably about 0.833 mM phosphoric acid. In another embodiment, the binding buffer may comprise about 10% to 30% ethanol. in further embodiment, the binding buffer may comprise about 0.5 M to about 2 M guanidine hydrochloride. In a still further embodiment, the binding buffer may comprise about 0.1M to about 1M sodium citrate. Typically, the device 10 will comprise about 0.3-0.5 g of silicon carbide resin and about 0.5-1 ml of binding buffer.

In use, the device 10 can be used to rapidly concentrate a urine sample without the use of specialized equipment such as centrifuges, and accordingly, without the need for electricity. To concentrate a urine sample, the device 10 is opened by removing any lids or seals and urine is collected into the tube 12 either through urinating directly into the tube 12 or by transferring from an initial collection container. The device 10 is then closed, and the urine is mixed well with the analyte specific binding agent 20 and the binding buffer 22 contained in the tube 12 by shaking the device 10 and its contents. Once the binding agent 20 has settled into the collection reservoir 20, the device 10 is opened and the remaining liquid is removed by decanting. The liquid may also be removed through the use of a pipette, syringe, dropper or any other device capable of transferring liquid. The target analyte is now in concentrated form and bound to the binding agent and may be subjected to further processing including the addition of a preservation solution or a lysis solution,

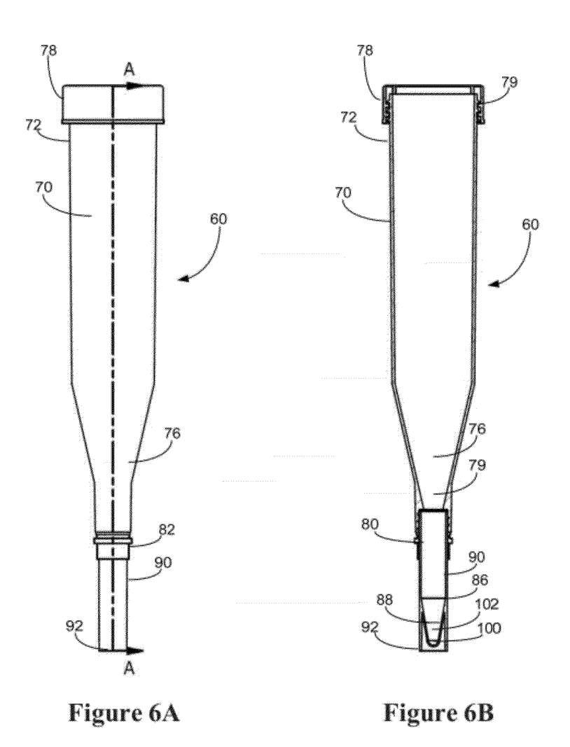

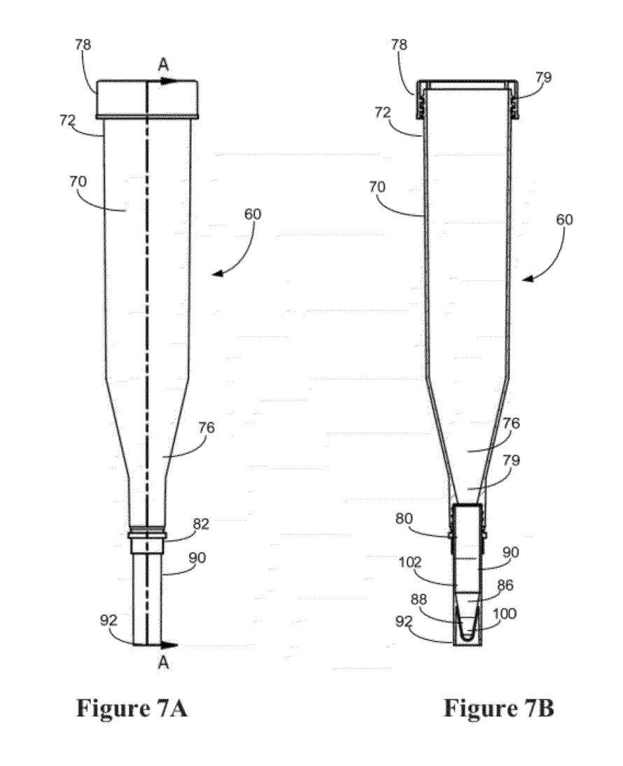

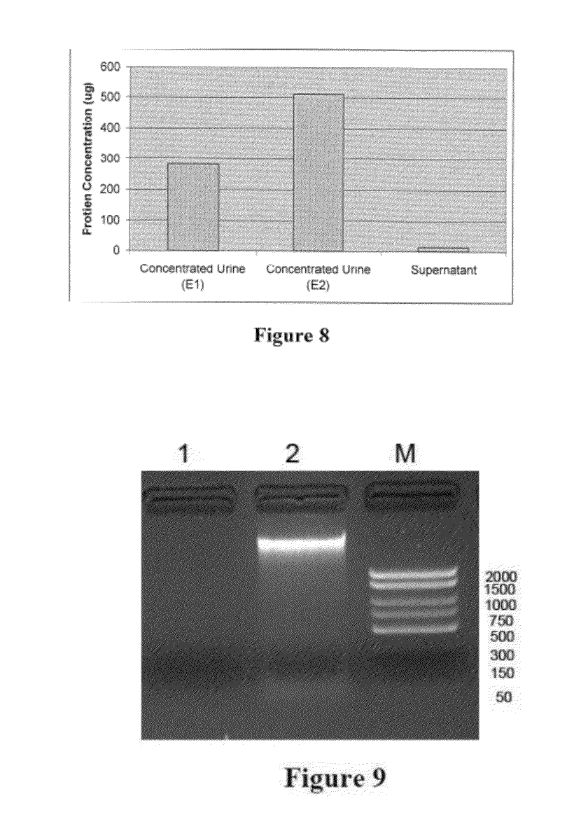

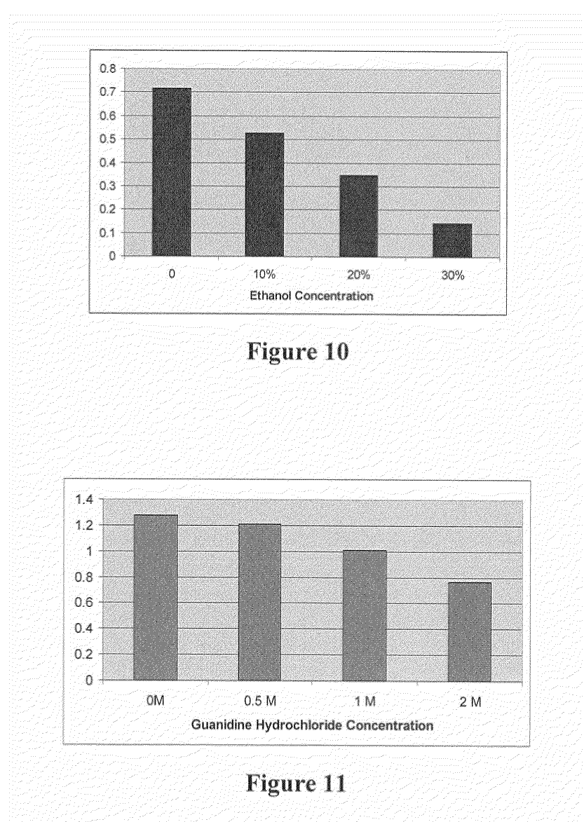

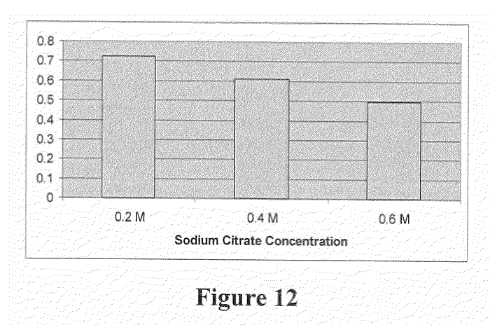

FIGS. 6A-B and 7A-B illustrate a second embodiment of a device 60 for the concentration of one or more target analytes contained in a urine sample. The device 60 comprises a first tube 70 and a second tube 80. The first tube 70 comprises an upper portion 72 defining an opening 74 for receiving the urine sample and a lower portion 76 for releasably attaching an upper portion 82 of the second tube 80. When the first tube 70 and the second tube 80 are attached to one another, the interior of said first tube 70 and the interior of said second tube 80 are in fluid communication allowing urine collected through the opening 74 in the first tube 70 to flow into the second tube 80 as shown in FIGS. 7A-B. in one embodiment, first tube 70 is larger than the second tube 80 to facilitate collection of the urine sample prior to concentration and shipping of the bound analytes in a reduced volume following concentration. The lower portion 76 of the first tube 32 and the upper portion 82 of the second tube 80 are provided with matching threads 79 and 84 respectively, whereby the lower portion 76 of the first tube 70 and the upper portion 82 of the second tube 80 can be screwed together to form a liquid tight seal.

As seen in FIGS. 6A-B and 7A-B, the second tube 80 may comprise an inner tapered portion 86 terminating in a collection reservoir 88 contained within an outer housing 90. The outer housing 90 may comprise a flat bottom edge 92 which allows the second tube 80 to stand freely without the use of an external support. The second tube 80 contains a predetermined amount of a binding agent 100 which specifically binds the one or more target analytes and of a predetermined amount of a binding buffer 102. The concentration device 60 may further comprise means for seating the opening of the first tithe 70 such as a screw on cap 78. While not shown in the figures, the smaller tube 80 may also be provided with means for sealing the opening Si of the second tube 80 following the detachment of the first tube 70. In one embodiment, the sealing means for both the first and second tubes may be screw on caps.

In a further embodiment, the second tube 80 can be substituted with the device 10 as described above (not shown). In such embodiments, the lower portion 76 of the first tube 32 and the upper portion 14 of the device 10 can be provided with matching threads, whereby the lower portion 76 of the first tube 70 and the upper portion 14 of the device 10 can be screwed together to form a liquid tight seal.

The size of device 60 will depend on the amount of urine to be collected. In a preferred embodiment, the opening of the first tube 70 can have an outer diameter of about 29.5 mm and an inner diameter of about 27 mm, The lower portion 76 of the first tube 70 tapers at the bottom and defines a bottom opening having outer diameter of about 13 mm and an inner diameter of about 9.9 mm. The first tube 70 can be about 149 mm in length. The second tube 80 can be about 47.5 mm in length. The opening of the second tube 80 can have an outer diameter of about 9.6 mm and an inner diameter of about 8.4 mm. The inner tapered portion 86 may be about 14.5 mm in length with the tapered portion 86 having an upper diameter of about 8.1 mm and the collection reservoir 88 having a diameter of about 4.6 mm. As discussed above, the choice of binding agent and binding buffer will depend on the desired urine analyte to be concentrated. in a preferred embodiment, the binding agent comprises silicon carbide and the binding buffer may comprise about 0.1 to 1 mM phosphoric acid, preferably about 0.5 mM phosphoric acid, and more preferably about 0.833 mM phosphoric acid. In another embodiment, the binding buffer may comprise about 10% to 30% ethanol, In further embodiment, the binding buffer may comprise about 0.5 M to about 2 M guanidine hydrochloride. In a stilt further embodiment, the binding buffer may comprise about 0.1M to about 1M sodium citrate. Typically, the concentration device may comprise about 0.3-0.5 g of silicon carbide and about 0.5-1 mL of binding buffer.

In use, the first and second tubes 70, 80 are attached together and urine is collected in the larger first tube 70 by either urinating directly into the tube or transferring the urine from another collection container. The first tube 70 is closed and the urine, binding agent 100 and binding buffer 102 are mixed well by shaking. The binding agent 100 with the bound analytes then settles rapidly by gravity into the smaller second tube. Once the binding agent 100 has settled, the cap 78 is removed and the liquid is decanted with the binding agent 100 with bound target analytes remaining at the lower tapered portion 86 of the smaller second tube 80. The liquid can also be removed through the use of a pipette, syringe, dropper or any other device capable of transferring liquid. The smaller second tube 80 can then be removed from the first tube 70 for further processing of the urine analytes, including adding a preservation solution or adding a lysis solution. The resulting preserved and/or lysed sample contained in the smaller tube can then be stored at room temperature and shipped at room temperature.

in a further aspect, disclosed is a kit for the collection and concentration of urine. The kit can be used to practice the methods disclosed herein. The kit may comprise either the single tube or double tube embodiments of the disclosed concentration devices along with printed instructions for use of the concentration device for concentrating urine analytes. The choice of the binding agent and the binding buffer comprising the concentration device will depend on the target analyte.

The kit may further comprise a pre-aliquoted, sealed ampule containing a preservation solution. In a preferred embodiment, the preservation solution is the NORGEN URINE PRESERVATIVE™ (Norgen's Urine Preservative Single Dose Ampules, Thorold, Canada, Cat#18124). The pre-aliquoted, sealed ampules may include, but are not limited to droppers, syringes, micro-dosers, pipettes, vials or pouches. The kit may further comprise a lysis solution.

Although the invention has been described with reference to illustrative embodiments, it is to be understood that the invention is not limited to these precise embodiments, and that various changes and modification are to be intended to be encompassed in the appended claims.

### EXAMPLE 1—CONCENTRATION OF URINE PROTEINS

A 50 mL sample of mid-stream urine was collected into a urine collection container, and 30 mL was then transferred into a urine concentration/shipping device (Norgen's Urine Concentration and Preservation Device, Thorold, Canada, Cat#38056) containing silicon carbide and Binding Buffer. The device was dosed and was inverted by hand several times in order to mix. The device was then placed upright and the silicon carbide is allowed to settle by gravity for 10 minutes. After the silicon carbide was settled, the top of the device was removed and the supernatant was decanted into a second 50 cc tube, ensuring that none of the silicon carbide was transferred with the supernatant. Next, 1 mL of Norgen Urine Preservative (Norgen's Urine Preservative Single Dose Ampules, Thorold, Canada, Cat#18124) was added to the silicon carbide and again the silicon carbide was mixed well by hand. The urine was then stored at room temperature for 1 hour prior to processing. After 1 hour, proteins were isolated from both the preserved urine sample and the decanted urine supernatant, Proteins were isolated from the preserved urine sample using Norgen's ProteoSpin Urine Protein Concentration Kit (Thorold, Canada, Cat#17400) and a modified procedure. Briefly, 500 μL. of Protein Wash Solution is added to the preserved urine sample, mixed well by hand, and settled by gravity for 10 minutes. The Wash Solution was removed using a 1 ml, micropipette and discarded. This wash step was then completed a second time, Next, 300 μL of Elution Buffer was added, mixed by inversion, and the silicon carbide settled by gravity. The elution (E1) was then removed using a I mL micropipette and transferred to an eppendorf tube containing 30 μL of Protein Neutralizer. The elution step was then repeated to obtain E2. To isolate proteins from the urine supernatant, 1 mL of the supernatant was processed as per Norgen's ProteoSpin Urine Protein Concentration Kit (Thorold, Canada, Cat#17400). To analyze the proteins present in both E1 and E2 from the concentrated and preserved urine sample, as well as from the urine supernatant, a Bradford Assay was performed and the results were graphed. As shown in FIG. 8, from the preserved urine sample, 283 μg of protein were recovered in E1 and 511 μg of protein were recovered in E2. Only 13 μg of protein was recovered from the supernatant, corresponding to less than 2% of the total proteins remaining in the supernatant after concentration using silicon carbide in the presence of an appropriate binding buffer,

### EXAMPLE 2—CONCENTRATION OF CELLS, BACTERIA AND DNA IN URINE

A 30 mL human urine sample was spiked with HEK 293 (10,000 cells/mL) and DH5α (10,000 cells/mL). The spiked urine sample was transferred into a urine concentration/shipping device (Norgen's Urine Concentration and Preservation Device, Thorold, Canada, Cat#38056) which contains silicon carbide and Binding Buffer. The device was closed and was inverted by hand several times in order to mix. The device was then placed upright and the silicon carbide was allowed to settle by gravity for 10 minutes. After the silicon carbide was settled, the top of the device was removed and the supernatant was decanted into a second 50 cc tube, ensuring that none of the silicon carbide was transferred with the supernatant. Next, 1 mL of Norgen Urine Preservative (Norgen's Urine Preservative Single Dose Ampules, Thorold, Canada, Cat#18124) was added to the silicon carbide and again the silicon carbide was mixed well by hand. The urine was then stored at room temperature for 1 hour prior to processing. After 1 hour, DNA was isolated from both the preserved urine sample and the decanted urine supernatant. To isolate DNA from the preserved sample, Norgen's Urine DNA Isolation Mini Kit (Thorold, Canada, Cat #27000) was used with a modified protocol. Briefly, Pronase and Proteinase K were added and the sample mixed well by hand. Next, 500 μL of Binding Solution was added, the sample mixed well by hand, and allowed to settle by gravity for 10 minutes. After settling the supernatant was removed using a 1 mL micropipette and discarded. Next, 500 μL of Wash Solution is added to the preserved urine sample, mixed well by hand, and settled by gravity for 10 minutes. The Wash Solution was removed using a 1 mL micropipette and discarded. Next, 500 μL of Wash Solution II is added to the preserved urine sample, mixed well by hand, and settled by gravity for 10 minutes. The Wash Solution was again removed using a 1 mL micropipette and discarded. Next 300 μL of Elution Buffer was added, mixed by inversion, and the silicon carbide settled by gravity. To isolate DNA from the urine supernatant, 1 mL of the supernatant was processed as per Norgen's Urine DNA Isolation Micro Kit (Thorold, Canada, Cat#18100). The purified DNA was then analyzed by running aliquots on a 1.5% agarose gel. As shown in FIG. 9, DNA was not visible in the lane corresponding to the urine supernatant, while large quantities of DNA could be seen in the lane corresponding to the concentrated urine sample. The results demonstrated that spiked HeLa cells and bacterial cells were bound to the silicon carbide and were concentrated within the urine. These results were further verified by performing real-time PCR amplification using primers specific for the 5S gene from HEK. 293 cells and the 16S gene from DH5α.

### EXAMPLE 3—BINDING OF LAM FROM URINE TO SILICON CARBIDE USING ETHANOL

Four different 1 mL urine samples were spiked with 5 pg/mL of lipoarabinomarman (LAM) and mixed well. Next, 100 mg of silicon carbide (grit size 2500) was added to the tubes containing the urine sample. Ethanol was then added to 3 of the tubes in order to allow the LAM to bind to the silicon carbide resin. The ethanol was added such that the final concentration of ethanol was 10% in the first tube, 20% in the second tube, and 30% in the third tube. The fourth tube was used as control and no ethanol was added. All the tubes were closed and mixed by inverting for 30 seconds. After mixing, the resin settled through gravity to the bottom of the tubes. The urine supernatant was then removed using a pipette and transferred to a clean tube. Next, 200 μL of water was added to the resin and mixed by inversion for 30 seconds in order to elute the bound LAM from the silicon carbide resin.

To test the effect of increasing ethanol concentration on binding LAM to silicon carbide, 100 μL of the urine supernatant that was removed from each tube after binding was subjected to the Clearview TB ELISA test, which uses antibodies specific to LAM.

As seen in FIG. 10, it was determined that LAM was indeed binding to the silicon carbide in the presence of ethanol, and that as the ethanol concentration increased the amount of LAM binding to the silicon carbide also increased as less LAM was present in the urine supernatant.

### EXAMPLE 4—BINDING OF LAM FROM URINE TO SILICON CARBIDE USING GUANIDINE HYDROCHLORIDE

Four different 1 mL urine samples were spiked with 10 pg/mL of lipoarabinomannan (LAM) and mixed well. Next, 100 mg of silicon carbide (grit size 2500) was added to the tubes containing the urine sample. Guanidine hydrochloride was then added to 3 of the tubes in order to allow the LAM to bind to the silicon carbide resin. The guanidine hydrochloride was added such that the final concentration of guanidine hydrochloride was 0.5M in the first tube, 1.0M in the second tube, and 2.0M in the third tube. The fourth tube was used as control and no guanidine hydrochloride was added. All the tubes were closed and mixed by inverting for 30 seconds. After mixing, the resin settled through gravity to the bottom of the tubes. The urine supernatant was then removed using a pipette and transferred to a clean tube. Next, 200 μL of water was added to the resin and mixed by inversion for 30 seconds in order to elute the bound LAM from the silicon carbide resin.

To test the effect of increasing guanidine hydrochloride concentration on binding LAM to silicon carbide, 100 μL of the urine supernatant that was removed from each tube after binding was subjected to the Clearview TB ELISA test, which uses antibodies specific to LAM.

As seen in FIG. 11, it was determined that LAM was indeed binding to the silicon carbide in the presence of guanidine hydrochloride, and that as the concentration of guanidine hydrochloride increased the amount of LAM binding to the silicon carbide also increased as less LAM was present in the urine supernatant.

### EXAMPLE 5—BINDING OF LAM FROM URINE TO SILICON CARBIDE USING SODIUM CITRATE

Three different 1 mL urine samples were spiked with 5 pg/mL of lipoarabinomannan (LAM) and mixed well. Next, 100 mg of silicon carbide (grit size 2500) was added to the tubes containing the urine sample. Sodium citrate was then added to the 3 tubes in order to allow the LAM to bind to the silicon carbide resin. The sodium citrate was added such that the final concentration of sodium citrate was 0.2M in the first tube, 0.4M in the second tube, and 0.6M in the third tube. All the tubes were closed and mixed by inverting for 30 seconds. After mixing, the resin settled through gravity to the bottom of the tubes. The urine supernatant was then removed using a pipette and transferred to a clean tube. Next, 200 μL of water was added to the resin and mixed by inversion for 30 seconds in order to elute the bound LAM from the silicon carbide resin.

To test the effect of increasing sodium citrate concentration on binding LAM to silicon carbide, 100 μL of the urine supernatant that was removed from each tube after binding was subjected to the Clearview TB ELISA test, which uses antibodies specific to LAM.

As seen in FIG. 12, it was determined that LAM was indeed binding to the silicon carbide in the presence of sodium citrate, and that as the sodium citrate concentration increased the amount of LAM binding to the silicon carbide also increased as less LAM was present in the urine supernatant.

### REFERENCES

Cook R L, Hutchinson S L, Ostergaard L, Braithwaite R S, Ness R B. Systemic review: Noninvasive tessting for Chlamydia trachomatis and Neisseria gonorrhoeae. Ann intern Med. 2005; 142:914-925

Chapin K C (2006) Molecular tests for detection of the sexually-transmitted pathogens Neisseria gonorrhoeae and Chlamydia trachomatis. Med Health RI 89: 202-204.

Zhang J, Tong K, Li P, Chan A, Yeung C, Pang C, et al. Presence of donor- and recipient-derived DNA in cell-free urine samples of renal transplantation recipients: urinary DNA chimerism. Clin Chem 1999; 45:1741-46.

Botezatu I, Serdyuk 0, Potapova G, Shelepov V. Alechina R, Molyaka Y, et. al. Genetic analysis of DNA excreted in urine: a new approach for detecting specific genomic DNA sequences from cells dying in an organism. Clin Chem 2000; 46:1078-84.

Le Lann A D, Bret L C, Delverdier M E, Amardeilh M F, Alary C, Fournie G J. Urinary DNA as an indicator of nephrotoxicity caused by endotoxin and gentamicin in mice. Toxicology 1994; 92:1-10.

Shigemura K, Shirakawa I, Okada H, Tanaka K, Kamidono S, Arakawa S. Gotoh A. Rapid detection and differentiation of gram-negative and gram-positive pathogenic bacteria in urine using Taqman probe. Clin Exp Med 2005; 4:196-201.

Exner M M, Lewinski M A. Isolation and detection of Borrelia burgdorferi DNA from cerebral spinal fluid, synovial fluid, blood, urine, and ticks using the Roche MagNA Pure system and real-time PCR. Diag Microbioi Infect Dis 2003; 46:235-40.

Prusty B K, Kumar A, Arora R, Batra S, Das B C. Human papillomavirus (HPV) DNA detection in self-collected urine, Int J Gynecol Obstetrics 2005;90:223-7

Paixoa P, Almeida S. Gouveia P, Binda S, Caroppo S, Barbi M. Diagnosis of congenital cytomegalovirus infection by detection of viral DNA in urine pools. J Virol Meth 2005:128:1-5.

Vanchiere J A, White Z S, Butel J S. Detection of BK virus and simian virus 40 in the urine of healthy children. J Med. Virol 2005; 75:447-54,

Pirtskalaishvili G, Konety B R, Getzengerg R H, Update on urine-based markers for bladder cancer. Postgrad Med 1999; 106:85-94.

Su Y, Wang M, Brenner D E, Ng A, Melkonyan H, Umansky S. et al. Human urine contains small, 150 to 250 nucleotide-sized, soluble DNA derived from the circulation and may be useful in the detection of colorectal cancer. J Mol Diagn 2004; 6:101-7.

Henrique R, Jeronimo C. Molecular detection of prostate cancer: a role for GSTP1 hypermethylation. Eur Urol 2004; 46:660-9,

Marques M A S, Damasceno L M P, Pereira H M G, Caldeira C M, Dias B F P, Vargens D G, et al. DNA typing: an accessory evidence in doping control. J Forensic Sci 2005; 50:1-6.

Cairns P (2004) Detection of promoter hypermethylation of tumor suppressor genes in urine from kidney cancer patients, Circulating Nucleic Acids in Plasma/Serum Iii and Serum Proteomics 1022: 40-43.

Mehta T K, Hoque M O, Ugarte R, Rahman M H, Kraus F, et al, (2006) Quantitative detection of promoter hypermethytation as a biomarker of acute kidney injury during transplantation. Transplantation Proceedings 38: 3420-3426

Cannas A, Goletti D, Giradi E, Chiacchio T, Calvo L, et al. (2008) Mycobacterium tuberculosis DNA Detection in Soluble Fraction of Urine from Pulmonary Tuberculosis Patients. International Journal of Tuberculosis and Lung Disease 12: 146-151

Fisa R, Riera C, Lopez-Chejade P, Molina I, Gallego M, et al. (2008) Leishmania infantum DNA detection in urine from patients with visceral Leishmaniasis and after treatment control. American Journal of Tropical Medicine and Hygeine 78: 741-744.

Mharakurwa S. Simoloka C, Thuma P E, Shiff C J, Sullivan D J (2006) PCR detection of Plasmodium falciparum in human urine and saliva samples. Malaria Journal 5: 103.

1. A device for the concentration of one or more target analytes contained in a urine sample, said device comprising:
a tube comprising an upper portion defining an opening for receiving the urine sample and a lower tapered portion terminating in a collection reservoir; said tube containing a predetermined amount of a particulate binding agent which specifically binds the one or more target analytes and of a predetermined amount of a binding buffer; and
means for sealing the opening of the tube.

2. The device according to claim 1, wherein said collection reservoir is substantially parallelepipedal in cross-section.

3. The device according to claim 2, wherein the collection reservoir comprises a tapered lower portion.

4. The device according to claim 3, wherein the tapered lower portion of the collection reservoir has a volume sufficient to contain the pre-determined amount of the particulate binding agent.

5. The device according to claim 1, wherein the binding agent comprises silicon carbide.

6. The device according to claim 1, wherein the means for sealing the opening of the tube is a screw on cap.

7. A device for the concentration of one or more target analytes contained in a urine sample, said device comprising:
a first tube and a second tube,
said first tube comprising an upper portion defining an opening for receiving the urine sample and a lower portion for releasably attaching an upper portion of the second tube; wherein when the first tube and the second tube are attached to one another, the interior of said first tube and the interior of said second tube are in fluid communication;
said second tube comprising a lower tapered portion terminating in a collection reservoir; said second tube containing a predetermined amount of a particulate binding agent which specifically binds the one or more target analytes and of a predetermined amount of a binding butler; and
means for sealing the opening of the first tube.

8. The device according to claim 7, wherein the binding agent comprises silicon carbide.

9. The device according to claim 7 further comprising means for sealing the opening of the second tube.

10. The device according to claim 9, wherein the means for sealing the opening of the second tube is a screw on cap.

11. The device according to claim 7, wherein the means for seating the opening of the first tube is a screw on cap.

12. The device according claim 7, wherein the lower portion of the first tube and the upper portion of the second tube are provided with matching threads, whereby the lower portion of the first tube and the upper portion of the second tube can be screwed together.

13. The device according to claim 7, wherein the said collection reservoir is substantially parallelepipedal in cross-section, wherein the collection reservoir comprises a tapered lower portion and wherein the tapered lower portion of the collection reservoir has a volume sufficient to contain the pre-determined amount of the particulate binding agent.

14. A method for concentrating one or more target analytes in a urine sample comprising the steps of:
a) providing a concentration device comprising a tube comprising an upper portion defining an opening for receiving the urine sample and a tower tapered portion terminating in a collection reservoir; said tube containing a predetermined amount of a particulate binding agent which specifically binds the one or more target analytes and of a predetermined amount of a binding buffer; and means for sealing the opening of the tube;
b) collecting a sample of urine into the concentration device;
c) mixing the urine sample, binding agent, and binding buffer;
d) allowing the binding agent to settle within the concentration device by gravity; and
e) removing the liquid portion of the urine sample from the concentration device, wherein the binding agent remaining in the concentration device is bound with the one or more target analytes.

15. The method of claim 14, wherein the binding agent comprises silicon carbide.

16. The method of claim 14, wherein the one or more analytes is DNA, RNA, microRNA, protein, bacteria, virus, yeast, exfoliated cells, polycyclic aromatic compounds and/or lipopolysaccharides.

17. The method of claim 14 further comprising the step of
f) adding a urine preservation solution to the binding agent bound with the one or more target analytes.

18. The method of claim 14 further comprising the step of:
g) adding a lysis solution to the binding agent bound with the one or more target analytes.

19. A kit for the collection and concentration of one or more target analytes from a urine sample; said kit comprising:
a concentration device comprising a tube comprising an upper portion defining an opening for receiving the urine sample and a lower tapered portion terminating in a collection reservoir; said tube containing a predetermined amount of a particulate binding agent which specifically binds the one or more target analytes and of a predetermined amount of a binding buffer; and means for sealing the opening of the tube; and
instructions for use of said concentration device.

20. The kit according to claim 19, further comprising a pre-aliquoted sealed ampule containing a preservation solution, wherein said pre-aliquoted sealed ampule is a dropper, a syringe, a micro-doser, a pipette, a vial, or a pouch.
